(19) United States
(12) Patent Application Publication
(10) Pub. No.: US 2021/0235777 A1
(43) Pub. Date: Aug. 5, 2021

(54) SUPPORT GARMENT FOR A PREGNANT WOMAN, AND METHOD FOR MANUFACTURING SAME

(71) Applicant: Sigvaris AG, St Gallen (CH)

(72) Inventor: Coline MESMIN, Saint Romain le Puy (FR)

(73) Assignee: Sigvaris AG, St Gallen (CH)

(21) Appl. No.: 17/268,487

(22) PCT Filed: Jun. 28, 2019
(86) PCT No.: PCT/EP2019/067315

§ 371 Date: Feb. 15, 2021

(30) Foreign Application Priority Data
Aug. 16, 2018 (FR) 1857513

(51) Int. Cl. A41C 1/10; A41D 1/21

(52) U.S. Cl. CPC A41C 1/10; A41D 1/21

(57) ABSTRACT

The invention concerns a support garment for a pregnant woman, comprising tights and a belt assembled together, the tights being a medical compression orthosis or everyday tights, characterized in that the belt comprises: a first band which extends around the waist in an uninterrupted manner between two joined ends and which is intended to come into contact with the skin, on the abdomen and the back of the pregnant woman, by putting it on or by employing adjustment means disposed at the two ends, and a second band made of a material that is less elastic than the first band and disposed on the first band as a reinforcement in order to support the weight of the forwardly swelling abdomen and thus relieve the back of the pregnant woman. The invention also concerns a method for the manufacture of a support garment for a pregnant woman.

### FIELD OF THE INVENTION

The present invention relates to the technical field of support garments for pregnant women, and more particularly to a garment formed by an assembly of everyday tights or medical compression tights, and a support belt.

### PRIOR ART

In this field, the document US2016/015089 describes various examples of garments comprising a belt and tights. The belt comprises a front portion and a back portion joined by side seams. The front portion comprises fabric portions with different thicknesses and/or elasticity. The back portion comprises at least one fabric portion made of a material which is similar to the front portion.

The disadvantage with that garment is the presence of side or ventral seams which produce a sensation of discomfort in contact with the skin, by squeezing the abdomen of the pregnant woman.

In addition, the side seams limit the support of the forwardly swelling abdomen, which could cause pain in the pregnant woman's back. These seams, weakened by the weight of the abdomen, could then loosen over time, deforming the belt or causing it to tear.

The number of layers of fabric constituting the belt also adds a difficulty when putting the belt around the pregnant woman's waist.

Finally, assembling the different portions of the belt involves high manufacturing costs and limits the possibility for providing a customized garment.

### DISCLOSURE OF THE INVENTION

The aim of the present invention is to propose an improved support garment for a pregnant woman which overcomes the above disadvantages.

To this end, a support garment for a pregnant woman has been developed, comprising tights and a belt assembled together, for example by means of an elastic seam. The tights are a medical compression orthosis or everyday tights,

In accordance with the invention, the belt comprises:
a first band which extends around the waist in an uninterrupted manner between two ends which are permanently or detachably joined, and which is intended to come into contact with the skin, on the abdomen and the back of the pregnant woman, by putting it on or by employing adjustment means disposed at the two ends, and
a second band made of a material that is less elastic than the first band, for example from polyurethane, and disposed on the first band as a reinforcement in order to support the weight of the forwardly swelling abdomen and thus relieve the back of the pregnant woman.

Thus, the invention can be used to provide a support garment for supporting the abdomen during pregnancy and which is practical, comfortable, aesthetic in design and inexpensive. In accordance with a first embodiment, the two ends of the first band are permanently joined by means of a single elastic seam. For better comfort, the seam is preferably disposed at the back of the pregnant woman. This seam may be vertical, for example, disposed in contact with the skin of the back along the spinal column. The back is also used as a support, allowing the belt to support the weight of the abdomen at the level of the seam and consequently to relieve the back of the pregnant woman. Thus, there is no risk that the stitching of the seam will wear or undo after the belt has been used once or just a few times.

In accordance with a second embodiment, the two ends of the first band are joined in a detachable manner via adjustment means, for example in the form of fastening tabs provided with touch and close systems, allowing the belt to be adjusted around the waist of the pregnant woman. In this configuration, positioning the belt around the waist is simple and rapid. In addition, the ability of the first band to adapt during the pregnancy comes from the pregnant woman adjusting the belt for her own comfort.

Preferably, the first band comprises a principal portion which is less elastic than an upper edge. In this manner, the principal portion can be used to provide the first band with a certain adaptability during the pregnancy (fundal height). In turn, the upper edge can maintain the support garment around the body of the pregnant woman.

Advantageously, the principal portion comprises fibres of elastane and fibres of at least one other material which is less elastic than the elastane, for example polyamide or polyester. The various fibres have a surface mass in the range 50 to 500 g/m². Preferably, the principal portion (33) has a surface mass in the range 200 to 300 g/m². In turn, the upper band comprises elastane fibres, for example.

Preferably, the fibres of the principal portion and of the upper edge are knitted together.

In accordance with a particular embodiment, the second band comprises holes forming a lacework. Thus, different patterns can be used in order to decorate the second band. in addition, the holes increase the elasticity of the second band, and thus improve the adaptability of the belt during the course of the pregnancy.

Another aim of the invention is to propose a simple and inexpensive method for the manufacture of the support garment, which can be adapted to the shape of the pregnant woman.

To this end, a method for the manufacture of a support garment for a pregnant woman has been developed, comprising tights and a belt assembled together in accordance with the foregoing.

In accordance with the invention, the method comprises the following steps:
cutting out the first band, designed to extend in an uninterrupted manner around the waist of the pregnant woman between its two ends which are meant to be joined;
cutting out the second band made of a material which is less elastic than the first band;
fastening the second band to the first band;
assembling the tights and the belt.

Thus, the simplified method can be used to reduce the costs of production while at the same time offering the pregnant woman the opportunity to have a tailored garment.

Preferably, the method employs an operation of heat fusible printing by pressing the second band onto the first band. This operation may be qualified as heat transfer or marking a textile with a thin film.

After the heat fusible printing operation, the belt may be rolled between two heated cylinders of a calendaring machine, for example with two passes of the belt between the cylinders, at a speed of 0.6 m/min and at a temperature of 175° C. In this way, the belt is more resistant to washing.

### BRIEF DESCRIPTION OF THE DRAWINGS

The invention will be better understood from the following description, which is given purely by way of non-limiting example and makes reference to the accompanying drawings, in which:

FIG. 1 is a front view of a pregnant woman wearing a support garment in accordance with a first embodiment of the invention;

FIG. 2 is a side view of the pregnant woman with the garment of FIG. 1;

FIG. 3 is a back view of the pregnant woman with the garment of FIG. 1;

FIG. 4 is a developed view of the first band constituting the belt of FIGS. 1 to 3;

FIG. 5 is a developed view of the belt of FIGS. 1 to 3, with the second band bonded to the first band by heat fusible printing;

FIG. 6 is a front view of a pregnant woman wearing a support garment in accordance with a second embodiment of the invention;

FIG. 7 is a side view of the pregnant woman with the garment of FIG. 6; and

FIG. 8 is a back view of the pregnant woman with the garment of FIG. 6.

### DETAILED DESCRIPTION OF THE INVENTION

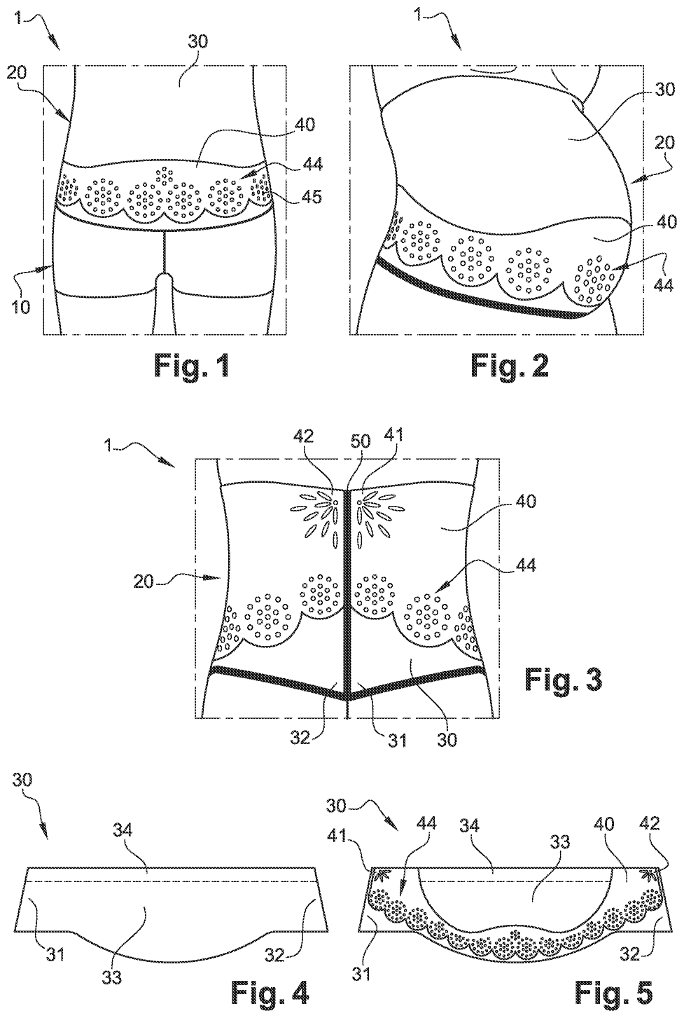

FIGS. 1 to 5 show a support garment (1) in accordance with the invention. The garment (1) comprises tights (10) and a belt (20) which are assembled together, for example by means of a horizontal elastic seam around the waist.

The tights (10) may be a medical compression orthosis, everyday tights, sport tights (compression or otherwise), or compression comfort tights (non-medical tights intended for individuals whose legs are healthy but who occasionally suffer from venous problems under certain situations, such as long aeroplane flights, pregnancy, being seated or standing for long periods at work). The tights (10) may be with feet, footless or with open feet. Preferably, the tights (10) are of the low rise type.

The belt (20) comprises two bands (30, 40) which are superimposed in the direction of their thickness. Each band (30, 40) is preferably single-layered. The band (40) may be fastened to the band (30) by heat fusible printing or by any other technique which is suitable for the envisaged application.

The lower band (30) is intended to be pulled on and come into contact with the skin on the pregnant woman's abdomen and back. The band (30) is cut in order out to fit the shape of the pregnant woman. The band (30) extends in an uninterrupted manner around the waist between two ends (31, 32) which are joined by means of an elastic seam (50) disposed vertically at the back.

The band (30) comprises a principal portion (33) and an upper edge (34), preferably constituted by elastic fibres which have been knitted together. Thus, the band (30) forms a single layer of material. The upper edge (34) is more elastic than the principal portion (33) and can be used to ensure that the garment (1) is held around the body of the pregnant woman.

Preferably, the upper edge (34) comprises solely fibres of elastane, while the principal portion (33) comprises fibres of elastane and of at least one other material which is less elastic than elastane, for example polyamide or polyester. Preferably again, the various fibres of the principal portion (33) have a surface mass which varies between 50 and 500 g/m². Even more preferably, the principal portion (33) has a surface mass in the range 200 to 300 g/m². Even more preferably, the band (40) has a thickness which is less than or equal to 180 μm.

The upper band (40) is disposed to reinforce the band (30), in order to support the weight of the forwardly swelling abdomen, and thus relieve the back of the pregnant woman. The band (40) forms an arc of a circle which extends in the lower portion of the band (30) over the front of the belt (20) in order to support the abdomen from below. In order to provide its reinforcement function, the band (40) is made of a material which is less elastic than the band (30). The band (40) is a film or a transfer forming a single layer of material. Preferably, the band (40) is produced from polyurethane. More preferably, the band (40) has a thickness which is less than or equal to 180 μm.

As can be seen in FIGS. 3 and 5, the band (40) extends in an uninterrupted manner between two adjacent ends (41, 42) of the two ends (31, 32) of the band (30). This provides better dorsal and ventral support.

The band (40) comprises holes (45) made through its thickness, arranged in patterns forming a lacework (44). The holes (45) can be used to improve the elasticity and the aesthetic appearance of the belt (30).

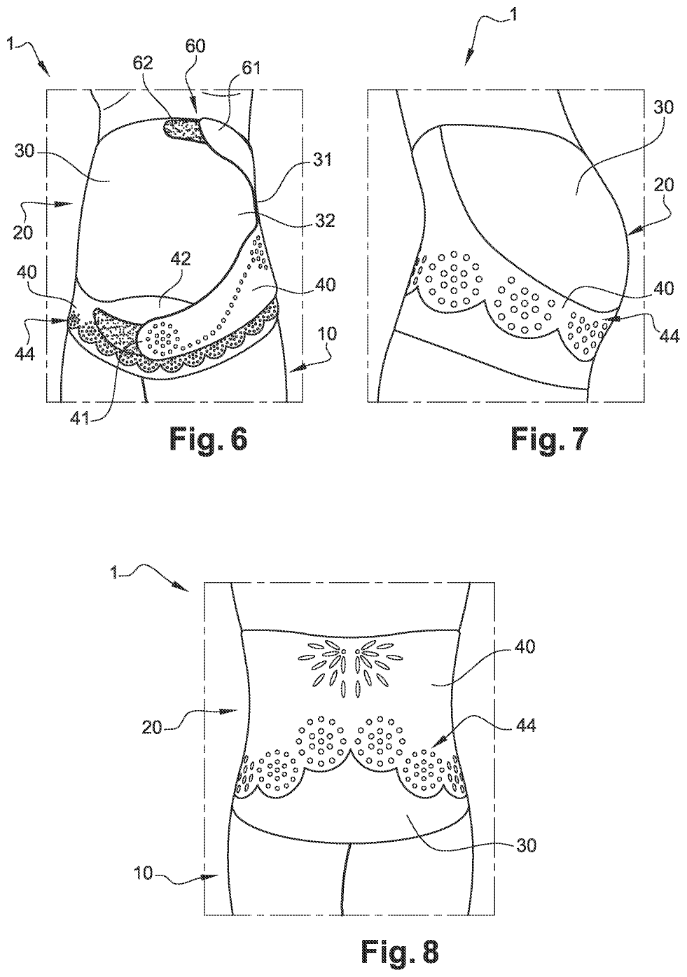

FIGS. 6 to 8 show a support garment (1) in accordance with a second embodiment of the invention.

Certain constituent elements of this garment (1) are comparable with those of the first embodiment described above and, for the purposes of simplification, have the same reference numerals.

In this second embodiment, the two ends (31, 32) of the band (30) are joined in a detachable manner by adjustment means (60) so that the belt (20) can be adjusted around the waist of the pregnant woman.

More precisely, the adjustment means (60) are touch and close type systems, for example. Thus, fastening tabs (61) formed at the end (31) of the band (30) are equipped with hooks provided in order to become fastened to the “loop” portions (62) of the belt (20) disposed on the side of the end (32). The tabs (61) of the band (30) form a “C”, for example, in order to enable fastening around the abdomen of the pregnant woman at the top and at the bottom. The tab (61) which will be fastened to the lower abdomen is also reinforced by the band (40) over its outer surface.

Thus, adjusting the belt (20) around the waist ensures that the lower abdomen is supported during pregnancy.

As an alternative, the adjustment means (60) may comprise fastening clips, or any other system which is suitable for the envisaged application.

Furthermore, the garment (1) may have a different shape to that of FIGS. 1 to 8 without departing from the scope of the invention.

Furthermore, some or all of the technical features of the various embodiments and variations mentioned above may be combined together.

Thus, the garment (1) can be adapted in terms of cost, features and performance.

It will be seen from the foregoing that the invention provides a support garment (1) for supporting the abdomen during pregnancy that is practical, comfortable, aesthetically pleasing and inexpensive.

1. A support garment for a pregnant woman, comprising tights and a belt assembled together, the tights being a medical compression orthosis, everyday tights, sport tights or comfort compression tights, characterized in that the belt comprises:
a first band which extends around the waist in an uninterrupted manner between two ends and which is intended to come into contact with the skin, on the abdomen and the back of the pregnant woman, by putting it on or by employing adjustment means disposed at the two ends, and
a second band made of a material that is less elastic than the first band and disposed on the first band as a reinforcement in order to support the weight of the forwardly swelling abdomen and thus relieve the back of the pregnant woman.

2. The support garment as claimed in claim 1, wherein the first band comprises a principal portion and an upper edge which is more elastic than the principal portion and which can ensure that the garment is held around the body of the pregnant woman.

3. The support garment as claimed in claim 2, the principal portion and the upper edge are constituted by elastic fibres knitted together.

4. The support garment as claimed in claim 2, wherein the principal portion comprises fibres of elastane and fibres of at least one other material which is less elastic than the elastane, the various fibres having a surface mass in the range 50 to 500 g/m².

5. The support garment as claimed in claim 2, wherein the upper edge solely comprises elastane fibres.

6. The support garment as claimed in claim 1, wherein the second band is made of polyurethane.

7. The support garment as claimed in claim 1, wherein the second band comprises holes forming a lacework.

8. The support garment as claimed in claim 1, wherein the two ends of the first band are permanently joined by means of a single elastic seam.

9. The support garment as claimed in claim 1, wherein the two ends of the first band are joined in a detachable manner via adjustment means allowing the belt to be adjusted around the waist of the pregnant woman.

10. A method for the manufacture of a support garment for a pregnant woman, comprising tights and a belt assembled together, the garment being as claimed in claim 1, wherein the method comprises the following steps:
cutting out the first band, designed to extend in an uninterrupted manner around the waist of the pregnant woman between its two ends which are meant to be joined;
cutting out the second band made of a material which is less elastic than the first band;
fastening the second band to the first band;
assembling the tights and the belt.
